(19) United States
(12) Patent Application Publication
(10) Pub. No.: US 2022/0047765 A1
(43) Pub. Date: Feb. 17, 2022

(54) UV Air and Surface Disinfection Apparatus

(71) Applicant: SEBI Environmental Services Inc., Newburgh, NY (US)

(72) Inventors: Marco Anthony D'Antonio, Amenia, NY (US); Sean Michael Morgan, Pine Bush, NY (US)

(21) Appl. No.: 17/401,245

(22) Filed: Aug. 12, 2021

Related U.S. Application Data
(60) Provisional application No. 62/706,386, filed on Aug. 13, 2020.

(51) Int. Cl. A61L 9/20; A61L 2/10

(52) U.S. Cl. CPC A61L 9/20; A61L 2/10; A61L 2202/11; A61L 2202/122; A61L 2209/15; A61L 2202/14; A61L 2209/11; A61L 2209/12

(57) ABSTRACT

An UV air and surface disinfection apparatus comprises a housing with moveable or adjustable walls, an air chamber defining a passageway operable to receive air and direct the air in a predetermined direction, a plurality of ultraviolet light sources supported by the housing and arranged in a predetermined pattern around the air chamber such that, when the plurality of ultraviolet light sources are energized, the plurality of ultraviolet light sources project ultraviolet radiation outward to facilitate sterilizing air as it moves through the air chamber and when the walls are open, the ultraviolet radiation is further projected outwardly to an environment or area outside of the housing to facilitate sterilizing surfaces.

This application claims benefit of U.S. Provisional application Ser. No. 67/706,386, filed on Aug. 13, 2020, and herein incorporated by reference in its entirety.

### (1) TECHNICAL FIELD

This disclosure relates to a disinfection apparatus employing ultraviolet radiation, and more particularly, to an apparatus effective to disinfect both air and surfaces.

### (2) BACKGROUND

The ability for germs to be transmitted from person to person is well known and transfer from animals to people has been documented. Some viruses and bacteria can persist on surfaces for days and remain airborne for hours. Recently, there has been increased interest in surface and air disinfection, particularly during times of infection or pandemic. There has also been interest in providing air disinfection, as airborne bacteria and other pathogens have been shown to cause hospital-acquired infections. Typically, the transmission of germs from one person to another is by accident. For example, some residential, commercial, or educational facilities have scores of people entering their facility each day. An individual entering the facility may be contagious with a pathogen and, in fact, may carry various strains of germs and/or viruses. The contagious individual may contaminate surfaces and may generate airborne particles. Uninfected persons in the facility may be exposed to pathogens by contacting the contaminated surfaces and by breathing in the airborne pathogen particles.

Various products, such as sanitization solutions, and techniques, such as frequent hand washing, are known to help prevent the spread of viruses. Protection from exposure of family, clients, or workers may be provided by gloves, masks, and disinfection units. Gloves may not be a practical option due to heat, loss of dexterity, or allergic reaction. Moreover, the gloves protect only the wearer. Masks may limit exposure but cannot be worn by everyone due to health issues, age, or inconvenience. In some cases, the mask may support bacterial growth. Some disinfection devices only provide surface disinfection but do not treat air or provide air disinfection and do not treat surfaces. There have been several devices developed which use ultraviolet C (UV-C) and have shown success in decontaminating surfaces within a room. Devices typically consist of multiple UV-C lamps arranged in a circular pattern and projecting UV-C radiation onto exposed surfaces of a room. These devices are typically mobile and can be wheeled into position as needed. UV-C surface disinfection units are not safe to use while the area is occupied. Several air purification systems have been developed to address this issue. Air disinfection units only treat the air passing through the device. Some air disinfection units can harm exposed individuals. HVAC units must be large to effectively treat the airflow and the blower fans must run continuously. Also, off the shelf UV systems designed for HVAC may not apply proper contact time because of the high velocity of air flow.

As can be seen, there is a need for an ultraviolet disinfection device which is capable of simultaneously and efficiently disinfecting both air and surfaces within a room or just disinfecting air while individuals are still in the room, using the same UVC source.

### SUMMARY

A principal object of the present disclosure is to disinfect air when a person is present while not exposing skin or eyes to UV light.

Another objective of the present disclosure to disinfect air and surfaces when the space is not occupied.

Still another objective is to allow changes of modes of treatment and duration of treatment responsive to site specific conditions.

Yet another objective of the invention is to employ ultraviolet lamps that produce ozone and/or ultraviolet lamps that do not produce ozone.

Yet still another objective of the invention is to provide one or more protection/operation devices such as an open door sensor, a motion sensor kill switch, a lamp intensity sensor to safely determine operation of the ultraviolet lamps, a low power notification, an error notification, a time interlock to ensure sufficient light dosage, an ozone concentration sensor, a tip sensor, and a safety interlock on light cover(s) when performing air only disinfection.

Yet another objective is operability under several modes and durations.

Another objective of the invention is to provide an ultraviolet light treatment unit that operates on various voltages.

In accordance with the objects of the disclosure, a UV air and surface disinfection apparatus is achieved. The UV air and surface disinfection apparatus comprises a housing with moveable or adjustable walls, an air chamber defining a passageway operable to receive air and direct the air in a predetermined direction, a plurality of ultraviolet light sources supported by the housing and arranged in a predetermined pattern around the air chamber such that, when the plurality of ultraviolet light sources are energized, the plurality of ultraviolet light sources project ultraviolet radiation outward to facilitate sterilizing air as it moves through the air chamber and when the walls are open, the ultraviolet radiation is further projected outwardly to an environment or area outside of the housing to facilitate sterilizing surfaces.

Also in accordance with the objects of the disclosure, an air-surface disinfector is achieved comprising an air chamber defining a passageway operable to receive air and direct air in a predetermined direction, baffling or an airflow interrupter for interrupting the air flowing through the air chamber, a housing enclosing the air chamber having an inlet end and an outlet end, a plurality of ultraviolet light sources arranged in a predetermined pattern such that when the plurality of ultraviolet light sources are energized, ultraviolet radiation is projected inwardly through the air chamber and outwardly to an environment or area around the plurality of ultraviolet light sources to facilitate sterilizing air within the disinfector, and one or more moveable or adjustable walls in the housing wherein when the walls are closed, the ultraviolet radiation is not allowed to project outside of the housing and wherein when the walls are open, the ultraviolet radiation is allowed to project outside of the housing to facilitate sterilizing surfaces outside of the disinfector.

Also in accordance with the objects of the disclosure, an air-surface disinfector is achieved comprising an air chamber defining a passageway for receiving air and for directing the air in a predetermined direction, the air chamber having an inlet end and an outlet end and a plurality of ultraviolet light sources arranged in a predetermined pattern such that when the plurality of ultraviolet light sources are energized, ultraviolet radiation is projected inwardly through the air chamber to facilitate sterilizing air passing through the air chamber and outwardly to facilitate sterilizing an environment or area around the plurality of ultraviolet light sources wherein the air chamber is not perforated and a blower or fan blows air in the inlet end through the air chamber and out the outlet end so that substantially all of the air entering the inlet end passes axially through the air chamber and is exposed to the ultraviolet radiation generated by the plurality of ultraviolet light sources, substantially not allowing the plurality of ultraviolet light sources to project ultraviolet radiation outward of the environment outside of the disinfector.

### BRIEF DESCRIPTION OF THE DRAWINGS

In the accompanying drawings forming a material part of this description, there is shown:

FIG. 1 is a bottom plan view of an ultraviolet light treatment unit according to a preferred embodiment of the present disclosure.

FIG. 2 is an electrical schematic depicting power routing in the preferred embodiment of the present disclosure.

FIG. 3 is a front elevation view of the preferred embodiment of the present disclosure showing the baffling system.

FIG. 4 is a top plan view of the ultraviolet light treatment unit according to a preferred embodiment of the present disclosure.

FIG. 5 is an oblique view of the preferred embodiment of the present disclosure in air only mode.

FIG. 6 is an oblique view of the preferred embodiment of the present disclosure in surface only mode or air and surface mode.

### DETAILED DESCRIPTION

The present invention provides a single disinfection unit utilizing ultraviolet lamps for localized surface treatment of surfaces, air treatment, or dual treatment. People and pets are preferably not harmed by the ultraviolet light C-band (UV-C) harmful rays. However, UV-C radiation is effective against bacteria, viruses, fungi such as mold spores, dust mites, and odors. Time delays and/or motion sensors may be used for dual or surface treatment when people or animals are not present. In air only mode, the unit may work safely in an environment without harm to the user. The device of the present disclosure is believed to be more cost effective than existing disinfection units and combines two technologies in one.

The following detailed description is of the best currently contemplated modes of carrying out exemplary embodiments of the disclosure. The description is not to be taken in a limiting sense, but is made merely for the purpose of illustrating the general principles of the disclosure, since the scope of the disclosure is best defined by the appended claims.

Broadly, an embodiment of the present disclosure provides a multifunction disinfection unit comprising housing, bulbs, ballasts, and controls. Preferably, the disinfection unit includes an ultraviolet light source. The unit may be opened for surface treatment and closed when the treatment area is occupied to continue treating air.

The housing may be a treatment container having a conventional defined shape. The interior of the housing may be constructed of aluminum, plastic or other materials, and may be coated with reflective paint. The light baffles may be coated with nonreflective paint. The materials may be selected due to the reflective properties (inside walls) and nonreflective properties (baffles) of the material which enhances operation of the ultraviolet light, but further provides a material of construction which inhibits growth of bacteria and lower virus activity duration. The sides may have doors which may be hinged, bolted, or louvered.

In some embodiments, the unit may further include one or more components selected from the group consisting of: ozone generating bulbs, light intensity indicator, ozone monitor, and automatic louver/door system.

In some embodiments, modifications may be made to one or more configurations selected from the group consisting of: fan placement, air flow direction, number of bulbs, spacing of bulbs, orientation of bulbs, wave length of UV light, size of unit, air flow rate, type of fan or blower, and changes to control logic.

In some embodiments, the unit may further provide one or more protection/operation devices selected from the group consisting of: an open door sensor, with or without a motion sensor kill switch or closing of mechanized doors; a lamp intensity sensor to safely determine operation of the ultraviolet lamps; a low power notification; an error notification; a time interlock to ensure sufficient light dosage; an ozone concentration sensor; a tip sensor; and a safety interlock on light cover(s) (e.g., when performing air only disinfection).

When the treatment area is unoccupied, the bulbs may be exposed to treat air and surfaces. When the treatment area is occupied, the bulbs may be covered, and airflow may be directed into the enclosure to continue to treat air. The ultraviolet lamp may activate manually, remotely, or under programmed time intervals. During this process, the air and/or surfaces are subjected to ultraviolet light for purposes of killing germs and/or viruses. Depending on the type of germicidal lamp employed, the ultraviolet light may also create an ozone residual to destroy germs that are hidden from the radiation light. A unit may be manufactured with both types of lamps to allow for both ultraviolet wavelengths and generation of ozone when required. Preferably, the units may be used in dual or surface mode when the area is unoccupied. During the time periods the area is occupied, preferably the unit is operated in air only mode.

To protect skin and eyes from UV radiation, the disinfection unit UV lamps may be cloaked, or the intensity may be adjusted manually or automatically.

The inventive unit may operate under several modes and durations. In some embodiments, the duration of treatment may be changed due to site specific conditions. The duration may be set for a time adjusted for the size of treatment area or required dosing of UV light. The unit may have remote start and/or a time delayed manual start.

In some embodiments, a user interface may be provided to prescribe unit usage type (e.g., air only or air/surface). The user interface may be located on the unit, or remotely. The unit may have “smart” unit capabilities with a smart interface. The unit may operate under air treatment only, surface only mode, or combined/dual (surface and air) treatment. In air-only mode, the unit may be safely run without exposing the user to any harmful eye or skin danger, since the unit will have a cover to block the user from direct exposure to harmful UV-C rays, yet allow air to flow across the lamps for disinfection and out the top of the unit, providing disinfected air without harmful germs and viruses.

In some embodiments, the unit may have remote monitoring capabilities and/or the unit may operate on various voltages.

UV bulbs/lamps may be surrounded by a quartz sleeve for each bulb/lamp. The ultraviolet lamps may be operated by a remotely located low voltage electrical source. The number and spacing of lamps may be dependent on the size of the treatment unit, airflow rate, or desired surface treatment radius. The units may vary in size to meet the needs of various users. The ultraviolet light emitted is preferably either at or around about 254 nm wavelength, capable of providing germicidal destruction, or about 185 nm, capable of producing radiant light and a residual amount of ozone. The unit may operate with either a single UV wavelength range or in a combination mode.

The disinfection unit may be assembled of readily available components and wired utilizing standard techniques. Structural components may be cut as required and fastened/secured using bolts, screws, or welding. Controls may either be programmable controllers or electromechanical relays or switches.

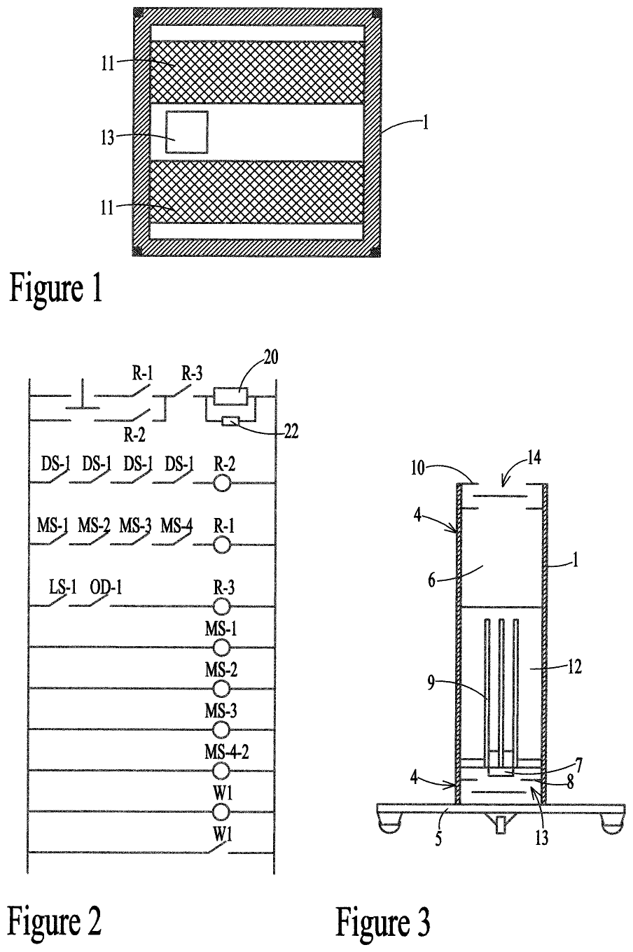

Referring now to FIGS. 1 to 6, an exemplary embodiment of the disclosed disinfection apparatus is shown. FIG. 1 is a view from the bottom of the apparatus and includes one or more ballast or transformers 11 which may be provided depending upon the type of lamps employed. An electrical ballast is a device placed in series with a load to limit the amount of current in an electrical circuit. Some ultraviolet emitting lamps including those that work on gas discharge exhibit negative resistance and so require the use of an external ballast to regulate current flow through the lamp.

FIG. 2 provides a wiring diagram, showing motion sensors MS-1, MS-2, MS-3, MS-4, level sensors LS-1, and ozone detectors OD-1 which may trigger a door switch DS-1 and ultraviolet light intensity failsafes UL-1. As shown in FIG. 2, a common voltage source such as 120 volts AC found in the home or business may be delivered to the treatment unit to power ultraviolet bulbs and/or capacitors 20 and fan 22. In air-only mode, upon closure of the door switches, electricity may be delivered to the lamps 20. In surface mode, motion sensors must not detect motion for the bulbs to receive power.

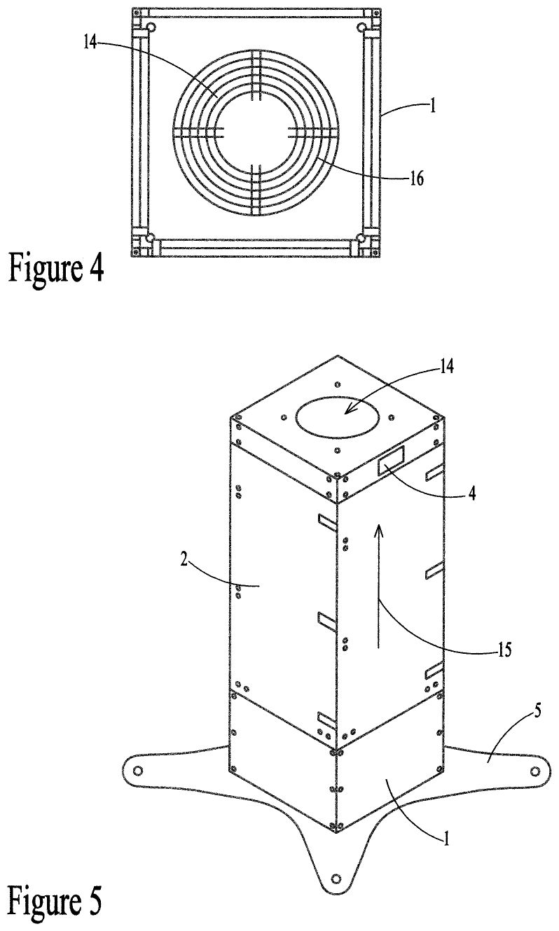

FIG. 4 illustrates a top view of an air outlet 14 having an end cap grate 16. The end cap grate 16 allows air to pass but does not allow light rays to pass. The air inlet on the opposite end of the apparatus, for example, 13 in FIG. 1, could also have an end cap grate on it.

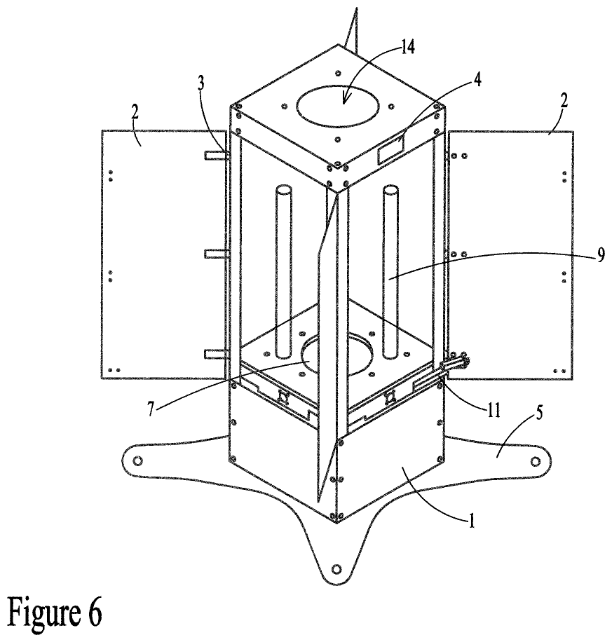

FIGS. 3, 5, and 6 show an exemplary housing 1 having an interior and removable, louvered, or openable side(s), wall(s), or door(s) 2. A side 2, with an inner surface and an outer surface, may be attached by a hinge member, latches, bolts, or other hardware 3 to the frame of the housing 1. The treatment unit may have alarm or indicator lights on control panel 4 to indicate when decontamination time has been met, when a door 2 is open, when motion is sensed, when the unit is tilted, or when light intensity is low. A control panel 4 may be on an upper surface as shown in FIGS. 5 and 6 and/or on a lower surface as shown in FIG. 3. The treatment unit may be set on ground (stationary), wall/floor/ceiling mounted, or may be mounted on a mobile wheeled base 5. In the interior of the housing, ultraviolet lamp(s) 9 may be positioned to provide effective distribution of ultraviolet light to air flow or surrounding surfaces. An additional retention time void 6 may be used to extend the duration that the air is in the treatment unit. The airflow may be generated by a unit blower or fan 7. A baffle system 8, 10 may be located below the fan and/or near the top of the unit to contain light during air-only mode. A switch on control panel 4 may engage the door 2 for operating in air only mode and allow operation of the lamps 9. Door automation 11 may be provided. Motion sensors on the control panel 4 may be provided to prevent ultraviolet light emission if humans or animals are present in the area. The air inlet 13 is located at the bottom of the unit and may be louvered or open. The opening may be on the bottom through the base 5 or on the sides of the air inlet 13. The air outlet 14 is at located at the top of the unit. The opening may be on the top or the sides of the air outlet, vertical or horizontal. FIG. 4 illustrates a preferred air flow direction 15. The air flow may be from the top, bottom, sides, or a combination; for example, inlet through a side of the apparatus and discharge through the top. If used, ballast or transformer 11 (shown in FIG. 1) will be at the bottom of the unit 1 in the area containing the baffles 8, shown in FIG. 3.

The invention is further represented in the following embodiments.

Embodiment 1. A disinfection unit comprising: a housing 1 with moveable or adjustable walls 2 with a blower or fan 7 for air movement.

Embodiment 1A. The disinfection unit of embodiment 1, further comprising an air chamber 12 operative to receive air blown by said blower or fan 7 when the disinfection unit is closed and for directing it in a predetermined direction, said chamber having an inlet end 13 and an outlet end 14.

Embodiment 1B. The disinfection unit of embodiment 1A, further comprising a plurality of ultraviolet light sources 9 supported by said housing 1 and arranged in a predetermined pattern around said chamber such that, when said plurality of ultraviolet light sources are energized, said plurality of ultraviolet light sources project ultraviolet radiation outward to facilitate sterilizing the air as it moves through the unit.

Embodiment 1C. The disinfection unit of one of embodiments 1, 1A, or 1B, wherein when in a dual air and surface mode or in a surface mode, walls 2 of the unit are open to an environment outside of the housing.

Embodiment 1 D. The disinfection unit of one of embodiments 1B or 1C, wherein said plurality of ultraviolet light sources 9 further project ultraviolet radiation through openings to surfaces to facilitate surface disinfection.

Embodiment 1E. The disinfection unit of one of embodiments 1C or 1 D, wherein said air chamber 12 is not perforated and said blower or fan 7 blowing air in said inlet end 13 through said air chamber 12 and out said outlet end 14 so that substantially all air entering said inlet end passes axially through said air chamber 12 and is exposed to said ultraviolet radiation generated by said plurality of ultraviolet light sources 9 substantially simultaneously as said plurality of ultraviolet light sources project ultraviolet radiation outward to facilitate sterilizing said environment outside said housing.

Embodiment 1F. The disinfection unit of one of embodiments 1B, 1C, 1D, or 1E, further comprising a controller coupled to said blower or fan 7 and said plurality of ultraviolet light sources 9 for controlling operation of the disinfection unit.

Embodiment 2. The disinfection unit of any one of embodiments 1D, 1E, or 1F, wherein said ultraviolet radiation is characterized by an intensity adapted to disinfect the surfaces.

Embodiment 3. The disinfection unit of any one of embodiments 1B, 1C, 1D, 1E, 1F, or 2, wherein said predetermined pattern comprises a generally circular pattern arranged around the air chamber.

Embodiment 4. The disinfection unit of any one of embodiments 1A, 1B, 1C, 1D, 1E, 1F, 2, or 3, wherein the air chamber is generally cylindrical or square.

Embodiment 5. The disinfection unit of any one of embodiments 1A, 1B, 1C, 1D, 1E, 1F, 2, 3, or 4, wherein the air chamber 12 is enclosed and baffled 8, 10 such that light does not escape the air chamber.

Embodiment 6. The disinfection unit of any one of embodiments, 1B, 1C, 1D, 1E, 1F, 2, 3, 4, or 5, further comprising a transparent tubular member, wherein the plurality of ultraviolet light sources are elongated and each has an axis that is generally parallel to an axis of said transparent tubular member.

Embodiment 7. The disinfection unit of any one of embodiments 1B, 1C, 1D, 1E, 1F, 2, 3, 4, 5, or 6, further comprising an end cap grate 13 through which allows air to pass but does not allow light rays to pass; said end cap being associated with first ends of said plurality of ultraviolet light sources 9 and adapted to retain said plurality of ultraviolet light sources in an upright and spaced relationship.

Embodiment 8. The disinfection unit of any one of embodiments 1, 1A, 1B, 1C, 1D, 1E, 1F, 2, 3, 4, 5, 6, or 7, wherein the disinfection unit is enclosed such that light does not emit from the unit.

Embodiment 9. The disinfection unit of any one of embodiments 6, 7, or 8, wherein the air chamber comprises a first end 13 situated on said housing 1 in operative relationship to said blower or fan 7, said blower or fan drawing air in from a base 5 of said housing 1 and expelling it through an outlet end 14 of said transparent tubular member.

Embodiment 10. The disinfection unit of any one of embodiments 1A, 1B, 1C, 1D, 1E, 1F, 2, 3, 4, 5, 6, 7, 8, or 9, wherein the predetermined direction 15 is vertical and upward.

Embodiment 11. The disinfection unit of any one of embodiments 1B, 1C, 1D, 1E, 1F, 2, 3, 4, 5, 6, 7, 8, or 9, further comprising at least one baffle or airflow interrupter 8, 10 operative to interrupt said air flowing through said air chamber 12.

Embodiment 12. The disinfection unit of any one of embodiments 6, 7, 8, 9, 10, or 11, wherein the plurality of elongated ultraviolet light sources 9 are lamps emitting C-band radiation.

Embodiment 13. A disinfection unit comprising: a housing 1 comprising a blower or fan 7 for blowing air; and an air chamber 12 operative to receive air blown by said blower or fan and to direct it in a predetermined direction.

Embodiment 13A. The disinfection unit of embodiment 13, further comprising a plurality of ultraviolet light sources 9 supported by said housing 1 and arranged in a predetermined pattern around said air chamber 12 such that, when said plurality of ultraviolet light sources are energized, said plurality of ultraviolet light sources project ultraviolet radiation outward through the housing and through said air chamber.

Embodiment 13B. The disinfection unit of embodiment 13A, further comprising a controller coupled to said blower or fan 7 and said plurality of ultraviolet light sources 9.

Embodiment 13C. The disinfection unit of one of embodiments 13, 13A or 13B, wherein said disinfection unit further comprises at least one baffle or airflow interrupter 8, 10 operative to interrupt air flowing through said air chamber 12.

Embodiment 14. An air-surface disinfector including an air chamber 12 defining a passageway for receiving air and for directing said air in a predetermined direction, comprising: a housing 1 and a plurality of ultraviolet light sources 9 arranged in a predetermined pattern such that, when said plurality of ultraviolet light sources are energized, ultraviolet radiation is projected inwardly through said air chamber and outwardly through said housing; wherein said air chamber has an inlet end 13 and an outlet end 14 and is not perforated; and a blower or fan 7 operative to blow air in said inlet end 13 and out said outlet end 14 so that substantially all of said air entering said inlet end passes axially through said air chamber 12 and is exposed to said ultraviolet radiation generated by said plurality of ultraviolet light sources.

Embodiment 15. An air disinfector comprising:

an air chamber 12 defining a passageway for receiving air and for directing said air in a predetermined direction, said air chamber having an inlet end 13 and an outlet end 14; and a plurality of ultraviolet light sources 9 arranged in a predetermined pattern such that, when said plurality of ultraviolet light sources are energized, ultraviolet radiation is projected inwardly through said air chamber to facilitate sterilizing air passing through said air chamber and outwardly to facilitate sterilizing an environment or area around said plurality of ultraviolet light sources;

wherein said air chamber is not perforated and a blower or fan 7 blows air in said inlet end 13 through said air chamber 12 and out said outlet end 14 so that substantially all of said air entering said inlet end passes axially through said air chamber and is exposed to said ultraviolet radiation generated by said plurality of ultraviolet light sources 9, substantially not allowing said plurality of ultraviolet light sources to project ultraviolet radiation outward of the environment outside of the unit.

Embodiment 16. The air-surface disinfector of embodiment 15, further comprising an end cap grate 13 through which air may pass; said end cap grate being associated with first ends of said plurality of ultraviolet light sources 9 and adapted to retain said plurality of ultraviolet light sources in an upright and spaced relationship and in operative relationship to said unit.

Embodiment 17. The air-surface disinfector of embodiment 15 or 16, wherein the air chamber 12 comprises a first end 13 situated on a housing 1 in operative relationship to a blower or fan 7, said blower or fan drawing air in from a base of said housing and expelling it through an exit opening 14 of said air chamber.

Embodiment 18. The air-surface disinfector of embodiment 15, 16, or 17, wherein the predetermined direction 15 is vertical and upward.

Embodiment 19. The air-surface disinfector as recited in claim 15, 16, 17, or 18, wherein said air-surface disinfector comprises baffling or an airflow interrupter 8, 10.

Embodiment 20. The air-surface disinfector as recited in claim 15, 16, 17, 18, or 19, wherein the plurality of ultraviolet light sources 9 are elongated lamps emitting C-band radiation.

Embodiment 21. An air-surface disinfector comprising:

an air chamber 12 defining a passageway operable to receive air and direct said air in a predetermined direction; and

a plurality of ultraviolet light sources 9 arranged in a predetermined pattern such that, when said plurality of ultraviolet light sources are energized, ultraviolet radiation is projected inwardly through said air chamber and outwardly to an environment or area around said plurality of ultraviolet light sources;

wherein said air-surface disinfector comprises baffling or an airflow interrupter 8, 10 for interrupting said air flowing through said air chamber.

It should be understood, of course, that the foregoing relates to exemplary embodiments of the disclosure, it is evident that many alternatives, modifications and variations would be, or are, apparent to those of ordinary skill in the applicable arts. Accordingly, Applicant intends to embrace all such alternatives, modifications, equivalents and variations that are within the spirit and scope of the disclosed subject matter and the appended claims.

What is claimed is:

1. An ultraviolet air and surface disinfection apparatus comprising:
a housing having one or more moveable or adjustable walls;
an air chamber defining a passageway operable to receive air and direct said air in a predetermined direction;
a plurality of ultraviolet light sources supported by said housing and arranged in a predetermined pattern around said air chamber such that, when said plurality of ultraviolet light sources are energized, said plurality of ultraviolet light sources project ultraviolet radiation outward to facilitate sterilizing air as it moves through said air chamber and when said walls are open, said ultraviolet radiation is further projected outwardly to an environment or area outside of said housing to facilitate sterilizing surfaces.

2. The apparatus according to claim 1 wherein when in a dual air and surface mode or in a surface mode, said walls are open to said environment outside of said housing and wherein when in an air only mode, said walls are closed to said environment outside of said housing.

3. The apparatus according to claim 1 further comprising a blower or fan wherein said air chamber is operative to receive air blown by said blower or fan and for directing said air in said predetermined direction, said air chamber having an inlet end and an outlet end;

4. The apparatus according to claim 3 wherein said air chamber is not perforated and wherein said blower or fan is configured to blow air in said inlet end through said air chamber and out said outlet end so that substantially all air entering said inlet end passes axially through said air chamber and is exposed to said ultraviolet radiation generated by said plurality of ultraviolet light sources substantially simultaneously as said plurality of ultraviolet light sources project ultraviolet radiation outward to facilitate sterilizing said environment outside said housing.

5. The apparatus according to claim 3 further comprising a controller coupled to said blower or fan and to said plurality of ultraviolet light sources for controlling operation of said disinfection apparatus.

6. The apparatus according to claim 1 wherein said ultraviolet radiation is characterized by an intensity adapted to disinfect surfaces.

7. The apparatus according to claim 1 wherein said predetermined pattern comprises a generally circular pattern arranged around said air chamber.

8. The apparatus according to claim 1 wherein said air chamber is generally cylindrical or square.

9. The apparatus according to claim 1 wherein said air chamber is enclosed and baffled such that light does not escape said air chamber.

10. The apparatus according to claim 1 wherein said ultraviolet light sources are ozone generating ultraviolet light bulbs or non-ozone generating ultraviolet light bulbs or a combination.

11. The apparatus according to claim 1 wherein said disinfection apparatus is enclosed such that light does not emit from said apparatus.

12. The apparatus according to claim 1 wherein said air chamber comprises a first end situated on said housing in operative relationship to a blower or fan, said blower or fan drawing air in from a base of said housing and expelling it through an outlet end of said air chamber.

13. The apparatus according to claim 12 further comprising an end cap grate which allows said air to pass but does not allow light rays to pass; said end cap being associated with first ends of said plurality of ultraviolet light sources and adapted to retain said plurality of ultraviolet light sources in an upright and spaced relationship, or said end cap is associated with said outlet end of said air chamber, or a combination.

14. The apparatus according to claim 1 wherein said predetermined direction is vertical and upward.

15. The apparatus according to claim 1 further comprising at least one baffle or airflow interrupter operative to interrupt said air flowing through said air chamber.

16. The apparatus according to claim 1 wherein said plurality of ultraviolet light sources are lamps emitting C-band radiation.

17. An air-surface disinfector comprising:
an air chamber defining a passageway operable to receive air and direct said air in a predetermined direction;
baffling or an airflow interrupter for interrupting said air flowing through said air chamber;
a housing enclosing said air chamber having an inlet end and an outlet end;
a plurality of ultraviolet light sources arranged in a predetermined pattern such that when said plurality of ultraviolet light sources are energized, ultraviolet radiation is projected inwardly through said air chamber and outwardly to an environment or area around said plurality of ultraviolet light sources to facilitate sterilizing said air within said disinfector; and
one or more moveable or adjustable walls in said housing wherein when said walls are closed, said ultraviolet radiation is not allowed to project outside of said housing and wherein when said walls are open, said ultraviolet radiation is allowed to project outside of said housing to facilitate sterilizing surfaces outside of said disinfector.

18. The disinfector according to claim 17 further comprising:
a blower or fan operative to blow air in said inlet end and out said outlet end so that substantially all of said air entering said inlet end passes axially through said air chamber and is exposed to ultraviolet radiation generated by said plurality of ultraviolet light sources.

19. An air-surface disinfector comprising:
an air chamber defining a passageway for receiving air and for directing said air in a predetermined direction, said air chamber having an inlet end and an outlet end; and
a plurality of ultraviolet light sources arranged in a predetermined pattern such that when said plurality of ultraviolet light sources are energized, ultraviolet radiation is projected inwardly through said air chamber to facilitate sterilizing air passing through said air chamber and outwardly to facilitate sterilizing an environment or area around said plurality of ultraviolet light sources;
wherein said air chamber is not perforated and a blower or fan blows air in said inlet end through said air chamber and out said outlet end so that substantially all of said air entering said inlet end passes axially through said air chamber and is exposed to said ultraviolet radiation generated by said plurality of ultraviolet light sources, substantially not allowing said plurality of ultraviolet light sources to project ultraviolet radiation outward of the environment outside of said disinfector.

20. The air-surface disinfector according to claim 19 further comprising an end cap grate through which air may pass, said end cap grate being associated with first ends of said plurality of ultraviolet light sources and adapted to retain said plurality of ultraviolet light sources in an upright and spaced relationship and in operative relationship to said disinfector, or said end cap is associated with said outlet end of said air chamber, or a combination.

21. The air-surface disinfector according to claim 19 wherein said air chamber comprises a first end situated on a housing in operative relationship to a blower or fan, said blower or fan drawing air in from a base of said housing and expelling it through an exit opening of said air chamber.

22. The air-surface disinfector according to claim 21 further comprising one or more moveable or adjustable walls wherein when said walls are closed, said ultraviolet radiation is not allowed to project outside of said housing and wherein when said walls are open, said ultraviolet radiation is allowed to project outside of said housing to facilitate sterilizing surfaces outside of said disinfector.

23. The air-surface disinfector according to claim 19 wherein said predetermined direction is vertical and upward.

24. The air-surface disinfector according to claim 19 further comprising baffling or an airflow interrupter.

25. The air-surface disinfector according to claim 19 wherein said plurality of ultraviolet light sources are elongated lamps emitting C-band radiation.
